(19) United States
(12) Patent Application Publication
(10) Pub. No.: US 2024/0200084 A1
(43) Pub. Date: Jun. 20, 2024

(54) AUTOTRANSPORTER SYSTEM

(71) Applicant: Prokarium Limited, London (GB)

(72) Inventors: Marc Biarnes Carrera, London (GB); Annelise Soulier, London (GB)

(21) Appl. No.: 18/010,792

(22) PCT Filed: Jun. 18, 2021
(86) PCT No.: PCT/GB2021/051561

§ 371 Date: Dec. 15, 2022

(30) Foreign Application Priority Data
Jun. 19, 2020 (GB) 2009411.6

(51) Int. Cl. C12N 15/74; A61K 38/20; A61K 38/21; A61K 39/00; A61K 39/39; A61K 45/06; C12R 1/42

(52) U.S. Cl. CPC C12N 15/74; A61K 38/20; A61K 38/21; A61K 39/39; A61K 45/06; A61K 2039/522; A61K 2039/523; C07K 2319/21; C07K 2319/43; C07K 2319/50; C12R 2001/42

(57) ABSTRACT

The present invention provides for a modified autotransporter and the use of genetically engineered microorganisms comprising said modified autotransporters in the treatment of infectious and neoplastic disease. The present invention therefore also relates to vaccine and immunotherapeutic compositions comprising said genetically engineered microorganism.

### CROSS REFERENCE TO RELATED APPLICATIONS

This application is a U.S. National Phase application filed under 35 U.S.C. § 371 and claims priority to International Application No. PCT/GB2021/051561, filed Jun. 18, 2021, which application claims priority to Great Britain Application No. 2009411.6, filed Jun. 19, 2020, the disclosure of which are incorporated herein by reference.

### FIELD OF INVENTION

The present invention relates to the modification of bacterial autotransporters in order to provide an optimal protein delivery system. The present invention also relates to genetically engineered microorganisms comprising the modified autotransporters herein disclosed and their subsequent use in the treatment of infectious disease and neoplastic disease.

### BACKGROUND OF INVENTION

Autotransporters, also referred to as AT or Type V secretion systems, are single genes that contain all the information required to cross both the inner membrane (IM) and outer membrane (OM) of Gram-negative bacteria. They contain an N-terminal signal peptide that targets the Sec secretion system (Green and Mecsas, 2016, Microbiology Spectrum, 4(1-19)), which has been shown to be important to prevent folding of protein in the periplasm (Szabady et al., 2005, PNAS, 102(221-226); followed by a “passenger” region, which is the functional region of the protein, linked to a beta-barrel (translocation unit, TU), used for transport through the OM, which can be aided by a β-helix structure known as the AutoChaperone domain (AC) (Renn et al., 2004, Biopolymers, 89(420-427), Velarde et al., 2004, Journal of Biological Chemistry, 279(31495-31504, Jong et al., 2012, Microbial Cell Factories, 11(1-11)). Several ATs also contain an extended β-helix in the passenger area, from which functional regions protrude (e.g., Hbp (Jong et al., 2012, Microbial Cell Factories, 11(1-11)). The passenger region is mostly cleaved, followed by possible re-anchoring in the bacterial surface through the beta-domain.

In the context of vaccine development using attenuated Salmonella or other bacteria, the display of heterologous antigens is normally accomplished by fusion of small epitopes (Jong et al., 2012, Microbial Cell Factories, 11(1-11), Jong et al., 2014, Microbial Cell Factories, 13(162)). For example, the AT MisL, from Salmonella enterica, involved in the survival of the bacterium in the gut by promotion of biofilm formation, was used to display epitopes of 8, 16 or 69 amino acids (Luria-Perez, 2007, Vaccine, 25(5071-5085), Mateos-Chavez et al., 2019, Frontiers in Immunology, 10(2562), Zhu et al., 2006, Vaccine, 24(3821-3831), Ruiz-Perez et al., 2002, Infection and Immunity, 70(3611-3620)).

These systems are attractive for their relative simplicity (i.e., only one gene being expressed) but can be influenced by some limitations, such as passenger size or presence of disulphide bonds. Furthermore, interactions between the heterologous protein and the C-terminus end of the AT may result in premature aggregation in the periplasm contributing to low yields.

Accordingly, there is a need in the field for an improved delivery system that can be used to deliver various different cargo in a way that overcomes the limitations described above.

### SUMMARY OF INVENTION

The inventors of the present invention have made the surprising discovery that modifying known autotransporters in the ways herein described produces an improved delivery system that can have various cargo rapidly introduced. It is a surprising discovery that such modifications result in an improved delivery system that overcomes the known limitations of autotransporters as a delivery system, such as premature aggregation of the protein to be delivered in the periplasm, therefore resulting in low yields and an inefficient process.

In a first aspect, the present invention provides an autotransporter construct modified to permit insertion of a heterologous polynucleotide sequence that encodes a target polypeptide for translocation across the inner and outer membrane of a Gram-negative bacterium, the autotransporter comprising i) a polynucleotide sequence that encodes a N-terminal signal sequence; ii) a passenger region into which said heterologous polynucleotide sequence encoding the target polypeptide is to be inserted and iii) a polynucleotide sequence encoding a translocation domain, wherein the passenger region comprises a synthetic polynucleotide sequence flanked by Type IIS restriction enzyme recognition sequences, wherein said synthetic polynucleotide sequence comprises a polynucleotide sequence that encodes a first polypeptide tag.

In a second aspect, the present invention provides for a genetically engineered microorganism comprising the autotransporter construct herein disclosed.

In a third aspect, the present invention provides for a vaccine composition comprising the autotransporter construct herein disclosed.

In a fourth aspect, the present invention provides for an immunotherapeutic composition comprising the autotransporter construct herein disclosed.

In a fifth aspect, the present invention provides for a vaccine composition or immunotherapeutic composition comprising the autotransporter construct herein disclosed for use in the prophylactic or therapeutic treatment of an infectious disease or a neoplastic disease.

In a sixth aspect, the present invention provides for a method for modifying a Gram-negative bacterial autotransporter, comprising: i) removing the passenger domain from the passenger region, ii) introducing a synthetic polynucleotide sequence that encodes a first polypeptide tag in the passenger region flanked by restriction enzyme recognition sequences, iii) introducing a synthetic polynucleotide sequence that encodes a second polypeptide tag within the passenger region upstream of the synthetic polynucleotide sequence that encodes the first polypeptide tag and is positioned outside of the restriction enzyme recognition sequence boundaries, iv) introducing a synthetic polynucleotide sequence that encodes a linker, said sequence being downstream of the synthetic polynucleotide sequence that encodes the first polypeptide tag and is positioned outside of the restriction enzyme recognition sequence boundaries, and v) introducing a synthetic polynucleotide sequence that encodes for a cleavage site within the passenger region downstream of the synthetic polynucleotide sequence that encodes for a linker.

### DESCRIPTION OF THE DRAWINGS

The invention is described with reference to the following drawings, in which:

FIG. 1 shows an exemplary schematic of a modified autotransporter (AT). Each AT may contain a wild-type cognate ribosome binding site (RBS), a signal-peptide allowing translocation of linear peptide to the periplasm, a BbsI-Golden Gate-compatible passenger-loading region, regions encoding passenger cleavage sites, and a translocation unit.

FIG. 2 shows an exemplary schematic of a cargo molecule comprising standardised 5′ and 3′ regions of DNA allowing for standard PCR amplification, and two BbsI restriction sites allowing rapid introduction of any cargo into the modified autotransporter.

FIG. 3 shows a schematic demonstrating the ampicillin survival challenge assay used herein using concentrated supernatant of each cultured strain.

FIG. 4 shows an exemplary agar plate from the ampicillin survival challenge assay herein used. A confluent lawn of ampicillin-sensitive (Amps) indicator strain (E. coli DH5α) was plated in Lysogeny Broth (LB) supplemented with 100 μg/mL of ampicillin. A volume of 1 μL of filter-sterilised 100× concentrated spent PCN media of each ZH9 strains expressing the indicated autotransporter (Hbp, EspP, EstA, AIDA-I, Pet, MisL) comprising the beta-lactamase (bla) gene as cargo. The plates were grown at 37° C. for 16-18 h.

FIG. 5 shows the final cell density of ampicillin-sensitive (Amps) indicator strain E. coli DH5a in 100 uL of Lysogeny Broth (LB) supplemented with increasing levels of ampicillin (ng/mL) and 1 μL of filter-sterilised 100× concentrated spent PCN media of each ZH9 strains expressing the indicated autotransporters (Hbp, EspP, EstA, AIDA-I, Pet, MisL) comprising the beta-lactamase (bla) gene as cargo in the presence of increasing levels of ampicillin (ng/mL). Supplement with extracts from Hbp, EspP, EstA, AIDA, and Pet allow growth of Amps indicator strain.

FIG. 6 shows the expression and secretion of the beta-lactamase cargo in the cell pellet and supernatant of the strains comprising various different modified autotransporters (Hbp, EspP, EstA, AIDA-b, Pet, MisL) via Western Blot analysis. The Bla protein is detected on the supernatant only in strains where the protein is fused to an autotransporter (e.g., EstaA or AIDA).

FIG. 7 shows the final cell density of ampicillin-sensitive (Amps) indicator strains (DH5α) in 100 μL of Lysogeny Broth (LB) supplemented with increasing levels of ampicillin (ng/mL) and 1 μL of periplasm protein extract (extracted by disruption with ice-cold water and Magnesium(II) supplement) of different ZH9 strains expressing the indicated autotransporters (Hbp, EspP, EstA, AIDA-I, Pet, MisL) either with or without the translocation unit (indicated as ΔTU where deleted), and having the beta-lactamase (bla) gene as cargo. The assay results show that deletion of the translocation unit does not prevent the cargo from localising into the periplasm of ZH9.

FIG. 8 shows the final cell density of ampicillin-sensitive (Amps) indicator strains (DH5α) in 100 μL of Lysogeny Broth (LB) supplemented with increasing levels of ampicillin (ng/mL) and 1 μL of filter-sterilised 100× concentrated spent PCN media of each ZH9 different ZH9 strains expressing the indicated autotransporters (Hbp, EspP, EstA, AIDA-b, Pet, MisL) either with or without the translocation unit (indicated as ΔTU where deleted), and having the beta-lactamase (bla) gene as cargo. The results show that deletion of the translocation unit does typically prevent export of cargo into the supernatant.

FIG. 9 shows exemplary SDS-PAGE gels and corresponding anti-6× Histidine Western Blots of protein present in the cell pellet (cytosol soluble and insoluble fraction, and periplasm), and supernatant of ZH9 Salmonella strains expressing autotransporter constructs where beta-lactamase (bla) is the passenger construct and with or without the translocation unit (where deletion is indicated as ΔTU). The results correlate with those from FIG. 8.

FIG. 10 shows exemplary SDS-PAGE gels and corresponding Western Blots of protein present in the concentrated spent PCN media after growth of ZH9 strains expressing the indicated autotransporters where the passenger region was loaded with either the V antigen from Yersinia pestis or the cytokine IL-18.

FIG. 11 shows final cell densities of ZH9 cultures expressing autotransporter constructs where beta-lactamase (bla) is the passenger construct. Results show that overexpression of autotransporters doesn't affect strain fitness.

### DETAILED DESCRIPTION

In order that the present invention may be more readily understood, certain terms are first defined. Additional definitions are set forth throughout the detailed description.

The term “autotransporter”, “AT” and “Type V secretion systems” are used interchangeably and refer to a family of outer membrane/secreted proteins (and corresponding polynucleotide sequence that encodes for said proteins) that have the ability to facilitate their own independent transport across the bacterial membrane system and final routing to the cell surface. Such proteins are known to have three key structural motifs; a signal sequence, a passenger domain, and a translocator domain. The term “modified autotransporter” therefore refers to an autotransporter that has been altered in some form, either by removing features or including new features, such that the modified autotransporter has an altered function when compared to the unmodified form. Preferably, the modified autotransporters herein disclosed are modified versions of autotransporters found in Gram-negative bacteria, in particular those found in Salmonella spp, E. coli or Pseudomonas spp. Particularly preferred autotransporters include EstA, MisL, Hbp, AIDA-1, EstP or Pet autotransporters, the unmodified sequences of which can be found in Yang et al., 2010, Journal of Biotechnology, 146(126-129) (EstA), Mateos-Chavez et al., 2019, Frontiers in Immunology, 10(2562) (MisL), Jong et al., 2012, Microbial Cell Factories, 11(85) (Hbp), Benz and Scmidt, 1992, Molecular Microbiology, 6(1539-1546) (AIDA-I), Skillman et al., 2005, Molecular Microbiology, 58(945-958) (EstP), or Sevastsyanovich et al., 2012, Microbial Cell Factories, 11(1-10) (Pet), the content of each of which is hereby incorporated by reference.

The term “passenger domain” refers to the N-terminal extracellular domain of an autotransporter. The passenger domain refers to the part of the autotransporter that encodes the protein to be exported and as such is variable in both length and sequence. The passenger domain of the modified autotransporters herein disclosed are particularly advantageous in that they allow for the efficient and reliable integration of various cargo into, for example, a microorganism, preferably, a Salmonella bacterium. The modified autotransporters herein disclosed allow for various cargos to be screened against the different modified autotransporters, for example, preferably those from Salmonella spp, E. coli or Pseudomonas spp, more preferably EstA, MisL, Hbp, AIDA-1, EstP or Pet, to determine the most efficient/reliable modified autotransporter to deliver the cargo in question. Accordingly, the present invention provides a way in which heterologous molecules can be efficiently and reliably delivered in a subject/patient based on the compatibility of the modified autotransporter/cargo, and therefore allow for an adaptive and flexible approach depending on the specific cargo to be delivered. As such, the modified autotransporters herein disclosed may be particularly useful in the field of oncology, where it is highly desirable to be able to deliver therapeutic molecules in a targeted way.

The term “signal sequence” refers to the N-terminal signal sequence of an autotransporter, the inclusion of which mediates the targeting pathway and translocation across the bacterial membrane. In the context of the present invention, such a signal sequence may mediate the translocation of a particular target peptide or protein across the bacterial membrane.

The term “polypeptide tag” refers to a synthetic peptide sequence that is commonly incorporated into an expression system. Such polypeptide tags can be used for purification, detection and localisation purposes.

As used herein, the term “attenuated”, in the context of the present invention, refers to the alteration of a microorganism to reduce its pathogenicity, rendering it harmless to the host, whilst maintaining its viability. This method is commonly used in the development of vaccines due to its ability to elicit a highly specific immune response whilst maintaining an acceptable safety profile. Development of such vaccines may involve a number of methods, examples include, but are not limited to, passing the pathogens under in vitro conditions until virulence is lost, chemical mutagenesis and genetic engineering techniques. Such an attenuated microorganism is preferably a live attenuated microorganism although non-live microorganisms are also disclosed.

By “genetically engineered microorganism” we mean any microorganism, for example, a bacterial (prokaryotic) cell, that has been genetically modified or “engineered” such that it is altered with respect to the naturally occurring cell. Such genetic modification may for example be the incorporation of additional genetic information into the cell, modification of existing genetic information or indeed deletion of existing genetic information. This may be achieved, for example, by way of transformation of a recombinant plasmid into the cell.

By “inactivating mutations”, we mean modifications of the natural genetic code of a particular gene or gene promoter associated with that gene, such as modification by changing the nucleotide code or deleting sections of nucleotide or adding non-coding nucleotides or non-natural nucleotides, such that the particular gene is either not transcribed or translated appropriately or is expressed into a non-active protein such that the gene's natural function is abolished or reduced to such an extent that it is not measurable. Thus, the mutation of the gene inactivates that gene's function or the function of the protein which that gene encodes.

The term “prophylactic treatment”, as used herein, refers to a medical procedure whose purpose is to prevent, rather than treat or cure, an infection or disease. In the present invention, this applies particularly to the vaccine composition. The term “prevent” as used herein is not intended to be absolute and may also include the partial prevention of the infection or disease and/or one or more symptoms of said infection or disease. In contrast, the term “therapeutic treatment” refers to a medical procedure with the purpose of treating or curing an infection or disease or the associated symptoms thereof, as would be appreciated within the art.

As used herein “heterologous polynucleotide” refers to a polynucleotide that has been introduced into the microorganism, for example the bacterium, i.e. the introduction of a polynucleotide that was not previously present. The polynucleotide may be exogenous to the bacterium, whereby these terms have their normal meaning in the art. For an endogenous polynucleotide, this may comprise introducing an additional copy or copies of said one or more endogenous polynucleotide in a heterologous manner. The endogenous polynucleotide or polynucleotides may also comprise introducing dominant variants of said polynucleotide or polynucleotides into the host bacterium, whereby “dominant” refers to the ability of the heterologous polynucleotide to functionally out-compete the naturally-occurring endogenous counterpart. The heterologous polynucleotide in the context of the present invention will encode for a target polypeptide intended for delivery i.e. for export and secretion, in a subject. The resulting polypeptide is also referred to herein as “cargo” or a “cargo molecule”.

The term “vaccine composition”, or “vaccine”, which from herein may be referred to interchangeably as the “composition”, relates to a biological preparation that provides active acquired immunity to a particular disease. Typically, the vaccine contains an agent, or “foreign” agent, that resembles the disease-causing pathogen. Examples of such a foreign agent may be a portion, or fragment, of a viral protein, capsule, DNA or RNA. Such a foreign agent would be recognised by a vaccine-receiver's immune system, which in turn would destroy said agent and develop “memory” against the disease-causing pathogen, inducing a level of lasting protection against future infection or disease from the same or similar pathogens. Through the route of vaccination, including those vaccine compositions of the present invention, it is envisaged that once the vaccinated subject again encounters the same pathogen or pathogen isolate of which said subject was vaccinated against, the individual's immune system may thereby recognise said pathogen or pathogen isolate and elicit a more effective defence against infection or disease. The active acquired immunity that is induced in the subject as a result of the vaccine may be humoral and/or cellular in nature.

By “immunotherapeutic composition”, we refer to any composition comprising an immunotherapeutic agent. Examples of immunotherapeutic compositions may also include additional components such as pharmaceutically acceptable carriers, biological response modifiers to enhance the immune response and/or adjuvants/excipients or diluents.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells or pathogens present as a result of an infectious disease.

A “checkpoint inhibitor” is an agent, which acts on surface proteins, which are members of either the TNF receptor or B7 superfamilies, including agents which bind to negative co-stimulatory molecules selected from CTLA-4, PD-1, TIM-3, BTLA, VISTA, TIGIT, LAG-3, and/or their respective ligands, including PD-L1.

The term “therapeutic antibody” as referred to herein includes whole antibodies and any antigen-binding fragment (i.e., “antigen-binding portion”) or single chains thereof which results in a therapeutic effect. Preferably, the therapeutic antibody is a monoclonal antibody, even more preferably, the therapeutic antibody and/or the monoclonal antibody may be a human or humanized antibody, the meaning of which will be readily understood by the skilled person.

By “cellular components of the immune system”, we refer to immunocytes such as lymphocytes, such as T and B lymphocytes gamma-delta T-cells, and NK cells, which may recognize specific antigens, such as prion, viral, bacterial, yeast, fungal, parasite, tumor-associated or tumor-specific antigens, or other antigens associated with a particular disease, disorder or condition. Other immunocytes include white blood cells, which may be granulocytes or agranulocytes. Examples of immunocytes include neutrophils, eosinophils, basophils, lymphocytes, monocytes, and macrophages. Dendritic cells, microglia, and other antigen-presenting cells are also included within this definition.

The terms “tumour,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations or regions within the subject, in which the sites, locations or regions are distinct from the primary tumour or cancer.

The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a disease, or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour. In reference to infectious disease, an effective amount may comprise an amount sufficient to reduce the viral load or to cause an improvement in the symptoms associated with said virus.

In some embodiments, a therapeutically effective amount is an amount sufficient to prevent or delay recurrence. A therapeutically effective amount can be administered in one or more administrations.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

The phrase “pharmaceutically acceptable” refers to molecular entities and compositions that do not produce an adverse, allergic or other untoward reaction when administered to a human, as appropriate. The preparation of a pharmaceutical composition that contains the vaccine composition or immunotherapeutic composition of the present invention will be known to those of skill in the art in light of the present disclosure. Moreover, for human administration, it will be understood that preparations should meet sterility, pyrogenicity, general safety and purity standards. A specific example of a pharmacologically acceptable carrier as described herein is borate buffer or sterile saline solution (0.9% NaCl).

As used herein, the term “subject” is intended to include human and non-human animals. Preferred subjects include human patients in need of enhancement of an immune response. The methods are particularly suitable for treating human patients having a disorder that can be treated by augmenting the immune response. In a particular embodiment, the methods are particularly suitable for treatment of cancer cells and infectious disease, for example, viral infections, in vivo.

As used herein, the terms “concurrent administration” or “concurrently” or “simultaneous” mean that administration occurs on the same day. The terms “sequential administration” or “sequentially” or “separate” mean that administration occurs on different days.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%. When particular values are provided in the application and claims, unless otherwise stated, the meaning of “about” should be assumed to be within an acceptable error range for that particular value.

In a first aspect, the present invention provides for an autotransporter construct modified to permit insertion a heterologous polynucleotide sequence that encodes a target for translocation across the inner and outer membrane of a Gram-negative bacterium, the autotransporter comprising i) a polynucleotide sequence that encodes a N-terminal signal sequence; ii) a passenger region into which said heterologous polynucleotide sequence encoding the target polypeptide is to be inserted and iii) a polynucleotide sequence encoding a translocation domain; wherein the passenger region comprises a synthetic polynucleotide sequence flanked by Type IIS restriction enzyme recognition sequences, wherein said synthetic polynucleotide sequence comprises a polynucleotide sequence that encodes a first polypeptide tag.

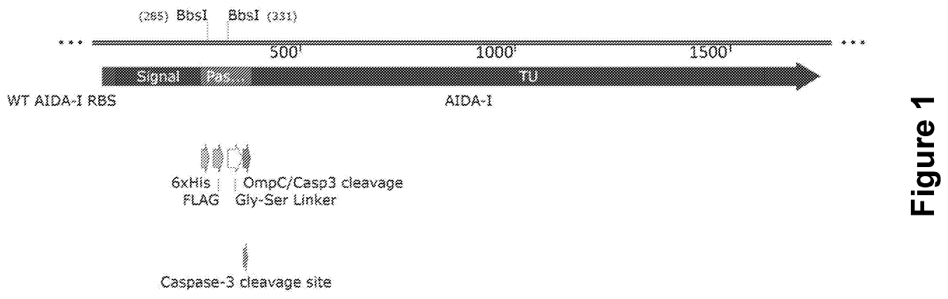

The modification of an autotransporter in the way described above allows for the creation of an improved delivery system in which a variety of different “cargo” can be rapidly introduced. Additionally, such a modification allows for different modified autotransporters, for example, modified versions of autotransporters found in Gram-negative bacteria, in particular those found in Salmonella spp, E. coli or Pseudomonas spp, for example, EstA, Hbp, AIDA-1, EstP or Pet, to be matched with the cargo based on their compatibility, and thus providing an improved/standardised delivery system that can be easily adapted to suit the need of the specific patient, opposed to assuming that a single autotransporter may be the optimum delivery source for all types of cargo. An exemplary modified autotransporter is shown in FIG. 1.

The polynucleotide sequence that encodes for the first polypeptide tag of the present invention may be any polynucleotide that can act as a placeholder until the cargo molecule can be introduced, and as such, has no functional effect. It is preferred that such a polynucleotide is of a shorter length so that any unwanted interaction with neighbouring segments is minimised. Therefore, the polynucleotide sequence that encodes for the first polypeptide tag may also be referred to as a neutral sequence or neutral region, and as such, does not encode for a biologically active molecule. By “shorter length” we intend for the first polypeptide tag sequence to be of a smaller size compared to the cargo sequence size, accordingly the precise length of the first polypeptide tag sequence is understood to be dependent on the size of the cargo to be exported. For example, it is known that the unmodified EstA autotransporter can export a cargo size of between approximately 20-60 kDa, the unmodified MisL autotransporter can export a cargo size of approximately 10 kDa, the unmodified Hbp autotransporter can export a cargo size of between approximately 5 and 60 kDa, the unmodified autotransporter AIDA-1 can export a cargo size of between approximately 5 and 70 kDa, the unmodified autotransporter EstP can export a cargo size of approximately 10 kDa and the unmodified autotransporter Pet can export a cargo size of approximately 5-110 kDa. Accordingly, depending on the cargo to be exported and the modified autotransporter to be used, the placeholder/neutral sequence herein disclosed will be shorter than the cargo sizes disclosed above. Whilst any polypeptide tag that fulfils the function described above may be suitable, in an embodiment, the first polypeptide tag sequence may be a FLAG tag having the sequence DYKDDDDK (SEQ ID NO: 1). Under certain circumstances, for example, in the context of pooled high-throughput cloning of cargo into autotransporters and evaluation of expression, such a polypeptide tag may be used to identify and discard cells that have failed to load the cargo. For example, following the introduction of various cargo into the modified autotransporters, protein expression of several single colonies may be used to assess the secretion using such polypeptide tags-if positive for the tag, for example, a FLAG tag, this is indicative that no loading of the cargo has occurred and the cells can be discarded. In another embodiment, the first polypeptide tag sequence may be any synthetic polynucleotide sequence that allows for the in-frame translation of the autotransporter. By “in-frame” we refer to the translation of nucleotides in the specific order that allows for the production of a particular protein. Where translation is not in-frame, a different protein is obtained. In the context of the present invention, an in-frame insertion allows for the correct translation of the Translocation Unit (located downstream of the passenger region). If it was not in-frame, for example, missing a nucleotide, the incorporated amino acids would not be the correct ones, resulting in a different polypeptide sequence and/or causing premature or delayed translation termination.

It is envisaged that the polynucleotide sequence encoding for the first polypeptide tag, for example, the FLAG tag, will be flanked by restriction recognition sites, and thus be recognised by restriction enzymes specific for those sites. The restriction recognition sites of the present invention may be recognised by Type I, Type II, Type III, Type IV or Type V restriction enzymes. In a preferred embodiment, the polynucleotide sequence encoding for the first polypeptide tag will be flanked by Type IIS restriction recognition sites, and therefore recognised by Type IS restriction enzymes specific for those sites, for example, BsaI, BsmBI or BbsI, having restrictions sites (5′ to 3′) of GGTCTCN (SEQ ID NO:2), CGTCTCN (SEQ ID NO:3) and GAAGACNN (SEQ ID NO:4) respectively. It is preferred that the length of the first polypeptide tag sequence when flanked by a recognition site is at least 6 base pairs. Preferably, Type IIS restriction enzymes are used, however, other restriction enzymes are not excluded. Type IIS restriction enzymes comprise a specific group of enzymes which recognise asymmetric DNA sequences and cleave at a defined distance outside of their recognition sequence, usually within 1 to 20 nucleotides. Such enzymes are widely used in cloning techniques such as Golden Gate cloning, which allow for high throughput cloning of the cargo. An example of a suitable Type IIS restriction enzyme for use in the present invention are BbsI, BsaI and BsmBI, preferably the restriction enzyme for use is BbsI.

The modified autotransporter construct herein disclosed may be further modified to comprise a synthetic polynucleotide sequence that encodes a linker. The linker allows for the isolation of the cargo from the translocation unit of the autotransporter. In a preferred embodiment, the linker sequence may be a serine-glycine linker, however, it is understood that any linker that achieves the desired effect may be suitable. As such, any short amino acid sequence that is able to act as a linker or “spacer” between the cargo and the translocation unit is herein disclosed. The linker may be between 1-100 amino acids in length, between 1-90 amino acids in length, between 1-80 amino acids in length, between 1-70 amino acids in length, between 1-60 amino acids in length, between 1-50 amino acids in length, between 1-40 amino acids in length, between 1-30 amino acids in length, between 1-20 amino acids in length, between 1-10 amino acids in length, between 1-5 amino acids in length, 10-100 amino acids in length, between 10-90 amino acids in length, between 10-80 amino acids in length, between 10-70 amino acids in length, between 10-60 amino acids in length, between 10-50 amino acids in length, between 10-40 amino acids in length, between 10-30 amino acids in length, between 10-20 amino acids in length, 20-100 amino acids in length, between 20-90 amino acids in length, between 20-80 amino acids in length, between 20-70 amino acids in length, between 20-60 amino acids in length, between 20-50 amino acids in length, between 20-40 amino acids in length, between 20-30 amino acids in length, 30-100 amino acids in length, between 30-90 amino acids in length, between 30-80 amino acids in length, between 30-70 amino acids in length, between 30-60 amino acids in length, between 30-50 amino acids in length, between 30-40 amino acids in length, 40-100 amino acids in length, between 40-90 amino acids in length, between 40-80 amino acids in length, between 40-70 amino acids in length, between 40-60 amino acids in length, between 40-50 amino acids in length, 50-100 amino acids in length, between 50-90 amino acids in length, between 50-80 amino acids in length, between 50-70 amino acids in length, between 50-60 amino acids in length, 60-100 amino acids in length, between 60-90 amino acids in length, between 60-80 amino acids in length, between 60-70 amino acids in length, 70-100 amino acids in length, between 70-90 amino acids in length, between 70-80 amino acids in length, 80-100 amino acids in length, between 80-90 amino acids in length or 90-100 amino acids in length. Examples of suitable linkers for use in the present invention are provided in Table 1 below (SEQ ID NOs 2-24), where the value in subscript represents the number of repeats of the sequence in brackets and the “/” represents where the linker is cleavable:

TABLE 1

| | | SEQ |
|---|---|---|
| | | ID |
| Linker | Type | NO |
| (GGGGS)₁₋₄ | Flexible | 5 |
| (G)₈ | Flexible | 6 |
| (G)₆ | Flexible | 7 |
| GSAGSAAGSGEF | Flexible | 8 |
| GGSAGGSGSGSSGGSSGASGTGTAGGTGSGSGT | Flexible | 9 |
| GSG | | |
| (EAAAK)₁₋₃ | Rigid | 10 |
| A(EAAAK)₄ALEA(EAAAK)₄A | Rigid | 11 |
| AEAAAKEAAAKA | Rigid | 12 |
| PAPAP | Rigid | 13 |
| (AP)₅₋₁₇ | Rigid | 14 |
| VSQTSKLTR/AETVFPDV | Cleavable | 15 |
| PLG/LWA | Cleavable | 16 |
| RVL/AEA | Cleavable | 17 |
| EDVVCC/SMSY | Cleavable | 18 |
| GGIEGR/GS | Cleavable | 19 |
| TRHRQPR/GWE | Cleavable | 20 |
| AGNRVRR/SVG | Cleavable | 21 |
| RRRRRRR/R/R | Cleavable | 22 |
| GFLG/ | Cleavable | 23 |
| GGSGGGSEGGGSEGGGSEGGGSEGGGSEGGGSG | Flexible | 24 |
| GGS | | |
| (GSSSSS)₁₋₉ | Rigid | 25 |
| (SSSSSS)₁₋₉ | Rigid | 26 |
| (GSSGSS)₁₋₉ | Flexible | 27 |

Preferred linkers are those that have flexible and soluble properties and are therefore comprised of small amino acids, such as serine and glycine. Suitable flexible linkers and their properties are further defined in Chen et al., 2013 (Adv Drug Deliv Rev, 65(10) and are herein incorporated by reference. The preferred linker sequence of the present invention is shown in SEQ ID NO: 25 below. Such a linker is preferred due to the lack of any repeating units and therefore less prone to undesired recombination events (Waldo et al., 1999, Nature biotechnology, 17 (691-695), although it is understood that the linkers disclosed in Table 1 will also suffice.

SEQ ID NO: 28:
GSAGSAAGSGEF

The synthetic polynucleotide sequence encoding for the linker herein disclosed may be located downstream of the synthetic polynucleotide sequence encoding for the first polypeptide tag and downstream of the restriction sites.

The autotransporter construct herein disclosed may further comprise a synthetic polynucleotide sequence that encodes a second polypeptide tag. The inclusion of a second polypeptide tag sequence enables the identification of the target protein from other similar-sized proteins, as well as a way in which the protein may be purified from a biological sample via affinity techniques. As such, the second polypeptide tag allows for an easy detection method to evaluate if the cargo is being secreted to the outside of the cell (i.e., supernatant). It also allows for the purification and concentration of protein in the outside media (i.e., supernatant) by using Ni-NTA columns, or other suitable methods, to allow a rapid qualitative detection and relative quantification (based on the relative comparison of densitometric intensity between two or more samples on a Western Blot or SDS-PAGE). Various polypeptide tags are able to fulfil this purpose, such tags include, but are not limited to; ALFA-tag, AviTag, C-tag, Calmodulin-tag, polyglutamate tag, EE-tag, FLAG-tag, HA-tag, His-tag, Myc-tag, NE-tag, Rho1 D4-tag, S-tag, SBP-tag, Spot-tag, Strep-tag, T7-tag, Ty tag, V5 tag, VSV-tag, Xpress tag. In a preferred embodiment, the second polypeptide tag sequence may encode for a His tag, also known as a polyhistidine tag. Such a tag consists of a string of histidine residues, ranging from four to ten residues, however it is preferred that a string of 6 histidine residues are used.

In some instances it is therefore possible that the first and second polypeptide tags may be the same, however, it is preferred that they are different in order that the purpose of one of the polypeptide tags does not interfere with the purpose of the second polypeptide tag.

The synthetic polynucleotide sequence encoding the second polypeptide tag may be located upstream of the synthetic polynucleotide sequence encoding the first polypeptide tag and upstream of the restriction sites.

In one embodiment, the autotransporter construct may further comprise a synthetic polynucleotide sequence that encodes a cleavage site. In a preferred embodiment, the cleavage site is a caspase-3 cleavage site and/or an OmpT cleavage site. Inclusion of such cleavage sites helps to ensure the secretion of the cargo being delivered by the modified autotransporter, opposed to the cargo being exposed on the surface only. The synthetic polynucleotide sequence encoding for the cleavage site may be located downstream of the synthetic polynucleotide sequence encoding for the first polypeptide tag, such that when in use the heterologous cargo is cleaved and separated from the other components of the autotransporter delivery system. It is particularly envisaged that the use of caspase-3 cleavage sites may be used to program cleavage of the “cargo” from the modified autotransporter inside of macrophages and/or to modify secreted cargos, for example, activation of enzymes, inside macrophages. This allows for the pre-programmed activation of recombinant immunostimulatory elements inside antigen presenting cells. Whilst caspase-3 cleavage sites are preferred, caspase-1 and caspase-11 cleavage sites may also be suitable for this purpose. Examples of suitable caspase-3 cleavage sites are provided for in SEQ ID NOs 29-37 below, further details of which can be obtained in Srikanth et al., 2010 (Science, 330: 390-393):

SEQ ID NO: 29:
DEVD
SEQ ID NO: 30:
DKAD
SEQ ID NO: 31:
DSFD
SEQ ID NO: 32:
DCTD
SEQ ID NO: 32
DGKD
SEQ ID NO: 34:
DYND
SEQ ID NO: 35:
DFRD
SEQ ID NO: 36:
DRPD
SEQ ID NO: 37:
DNVD

The synthetic polynucleotide sequence encoding for the cleavage site herein disclosed may be located downstream of the synthetic polynucleotide sequence encoding for the linker.

Optionally, the modified autotransporter herein disclosed may also comprise a ribosome binding site (RBS). For example, in a preferred embodiment, there is a wild-type cognate ribosome binding site. In an alternative embodiment, a synthetic RBS may be used.

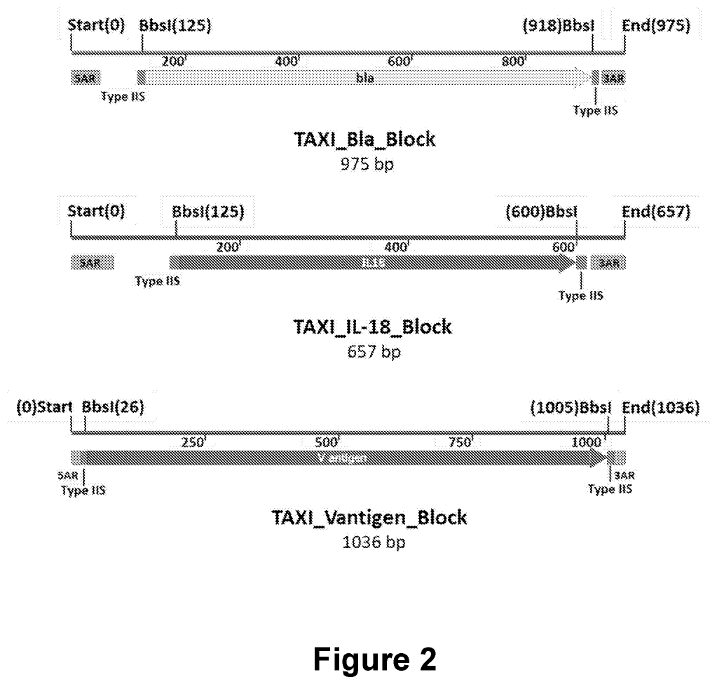

The present disclosure also provides for the design of a “cargo molecule” wherein a heterologous polynucleotide encoding the desired target protein or peptide, the “cargo”, can be rapidly cloned into the modified autotransporter of choice, for example, modified versions of autotransporters found in Gram-negative bacteria, in particular those found in Salmonella spp, E. coli or Pseudomonas spp, for example, EstA, Hbp, AIDA-1, EstP or Pet, using the Golden Gate technique (Engler et al., 2008, PLoS ONE, 3(11)), or any other similar technique that would be readily known to the skilled person. Said cargo molecule has designed nucleotides sequences located at the 5′ and 3′ of the heterologous polynucleotide (each end comprises a total of 4 bp that is complementary to the sequence within the vector, and upstream to these 4 bp is a section of “random” DNA, allowing for amplification of any cargo, using the same primers, and providing support to the restriction enzyme to cut properly) to allow for PCR amplification and subsequent cloning. An example of such a cargo molecule is provided in FIG. 2.

In an embodiment, the autotransporter to be modified may be modified autotransporters found in Gram-negative bacteria, in particular those found in Salmonella spp, E. coli or Pseudomonas spp, for example, EstA, MisL, Hbp, AIDA-1, EstP or Pet. EstA and MisL originate from Pseudomonas aeruginosa and Salmonella enterica respectively, whilst Hbp, AIDA-1, EstP and Pet originate from Escherichia coli. Details of said autotransporters are provided for in Table 2. As used herein, the term “cleavage” refers to an autotransporter that has a domain called “auto-catalytic”, which promotes self-cleavage of the passenger from the translocation unit. Said auto-catalytic domain is a particular amino acid sequence (dependent on the autotransporter) located between the passenger domain and the translocation unit that is spontaneously cleaved, resulting in release of the passenger from the translocation unit (which is attached at the bacterial cell membrane). The present invention allows for the modification of these autotransporters to allow the rapid introduction of a range of different sized cargos and efficient cleavage of said cargo into the modified autotransporter of choice. For example, the cargo may be between 1-100 kDa, between 1-90 kDa, between 1-80 kDa, between 1-70 kDa, between 1-60 kDa, between 1-50 kDa, between 1-40 kDa, between 1-30 kDa, between 1-20 kDa, between 1-10 kDa, between 1-5 kDa, 10-100 kDa, between 10-90 kDa, between 10-80 kDa, between 10-70 kDa, between 10-60 kDa, between 10-50 kDa, between 10-40 kDa, between 10-30 kDa, between 10-20 kDa, 20-100 kDa, between 20-90 kDa, between 20-80 kDa, between 20-70 kDa, between 20-60 kDa, between 20-50 kDa, between 20-40 kDa, between 20-30 kDa, 30-100 kDa, between 30-90 kDa, between 30-80 kDa, between 30-70 kDa, between 30-60 kDa, between 30-50 kDa, between 30-40 kDa, 40-100 kDa, between 40-90 kDa, between 40-80 kDa, between 40-70 kDa, between 40-60 kDa, between 40-50 kDa, 50- kDa, between 50-90 kDa, between 50-80 kDa, between 50-70 kDa, between 50-60 kDa, 60-100 kDa, between 60-90 kDa, between 60-80 kDa, between 60-70 kDa, 70-100 kDa, between 70-90 kDa, between 70-80 kDa, between 80-100 kDa, between 80-90 kDa or between 90-100 kDa. Accordingly, the present invention allows for an improved pairing of cargo and modified autotransporter to provide an efficient and reliable delivery system in a variety of therapeutic conditions. Additionally, the present invention allows for an easy method by which a variety of different modified autotransporters, for example, EstA, MisL, Hbp, AIDA-1, EstP or Pet, can be efficiently screened for their ability to export and secrete a chosen cargo. Whilst the above autotransporters are preferable, the modifications herein described may be applicable to any bacterial autotransporter.

TABLE 2

| | | Cargo Size | | | |
|---|---|---|---|---|---|
| | | (kDa) that the | | | |
| | | passenger | | | |
| | | region of the | | | |
| | | autotransporter | | | |
| Auto | | is capable of | | | B-coil |
| Transporter | Origin | exporting | | Cleavage | present? |
| EstA | Pseudomonas | 27-50 | kDa | No | No |
| | aeruginosa | | | | |
| MisL | Salmonella | ~10 | kDa | No | No |
| | enterica | | | | |
| Hbp | Escherichia coli | 9.9-30 | kDa | Yes | Yes |
| | | (total ~50 kDa) | | 1000-1001 | |
| AIDA-I | Escherichia coli | 6-63 | kDa | Yes | No |
| | | | | 846-847 | |
| EstP | Escherichia coli | 11 | kDa | Yes | Yes |
| | | | | 968-969 | |
| Pet | Escherichia coli | 10-105 | kDa | Yes | No |

1018-1019

In a preferred embodiment, the autotransporter construct may further comprise a heterologous polynucleotide encoding a target peptide or protein. Preferably, the heterologous polynucleotide encodes for any therapeutic protein suitable for delivery with the modified autotransporters herein disclosed. Preferably, the heterologous polynucleotide may encode for an anti-cancer therapeutic or immunogenic molecule, and as such be particularly useful in the oncology and/or vaccine fields. Accordingly, in an embodiment, the heterologous polynucleotide may trigger an immune response in a subject, i.e. be immunogenic. In yet a further preferred embodiment, the anti-cancer therapeutic or immunogenic molecule is a cytokine, a chemokine, an antibody or fragment thereof, a cytotoxic agent, a cancer antigen or any combination thereof. In one preferred embodiment, the heterologous polynucleotide may encode for both a cytokine and a cancer antigen.

The skilled person will readily understand that preferred combinations will be those where the two components work together to create a more efficacious effect, and/or those where one of the components supports the therapeutic action of the other. Such a target peptide or protein, or “cargo”, may, for example, be stimulatory molecules of human immune cells (e.g., cytokines, chemokines, cytotoxic agents), such as IFNβ, IFNγ and/or IL-18, antibodies, antibody fragments, polypeptides containing one or more epitopes to be used as vaccines, cytotoxic compounds. It is understood that any of the aforementioned cargo molecules, or combinations thereof, may be combined with any one of the autotransporters described above. For example, EstA may comprise a heterologous nucleotide encoding a cytokine, a chemokine, an antibody, an antibody fragment, a polypeptide containing one or more epitopes to be used as a vaccine, a cytotoxic compound, or any combination thereof. MisL may comprise a heterologous nucleotide encoding a cytokine, a chemokine, an antibody, an antibody fragment, a polypeptide containing one or more epitopes to be used as a vaccine, a cytotoxic compound, or any combination thereof. Hbp may comprise a heterologous nucleotide encoding a cytokine, a chemokine, an antibody, an antibody fragment, a polypeptide containing one or more epitopes to be used as a vaccine, a cytotoxic compound, or any combination thereof. AIDA-1 may comprise a heterologous nucleotide encoding a cytokine, a chemokine, an antibody, an antibody fragment, a polypeptide containing one or more epitopes to be used as a vaccine, a cytotoxic compound, or any combination thereof. EstP may comprise a heterologous nucleotide encoding a cytokine, a chemokine, an antibody, an antibody fragment, a polypeptide containing one or more epitopes to be used as a vaccine, a cytotoxic compound, or any combination thereof. Pet may comprise a heterologous nucleotide encoding a cytokine, a chemokine, an antibody, an antibody fragment, a polypeptide containing one or more epitopes to be used as a vaccine, a cytotoxic compound, or any combination thereof. In a second aspect, the present invention provides for a genetically engineered microorganism that comprises the modified autotransporter construct herein described. Accordingly, the present invention provides for a genetically engineered microorganism that comprises the heterologous polynucleotide encoding a target peptide or protein described above. The present invention also provides for a genetically engineered microorganism that comprises a modified autotransporter, wherein the autotransporter may be derived from a different microorganism to the microorganism being genetically engineered, for example, Pseudomonas aeruginosa, Salmonella enterica and Escherichia coli. Additionally, the present invention provides for a genetically engineered microorganism that comprises at least one of the modified autotransporter constructs herein described. Accordingly, the genetically engineered microorganism may have numerous, for example, 1, 2, 3, 4, 5 or 6, modified autotransporters (the same or different), each one of which encoding for the same cargo, or different cargos. It is understood that the precise number of modified autotransporters will be dependent on a number of factors, for example the desired purpose and/or the expression conditions. In one embodiment, the genetically engineered microorganism may have two modified autotransporters, as defined herein. In another embodiment, the genetically engineered microorganism may have three modified autotransporters, as defined herein. The skilled person will recognise that in such a situation, either numerous diseases can be targeted at once, or a single disease, for example, a specific cancer/neoplastic disease, can be targeted in a multifaceted way.

In one embodiment, the genetically engineered microorganism may be an attenuated bacterium, preferably wherein the genetically engineered bacterium is a Gram-negative bacterium. Examples of Gram-negative bacteria for use in the present invention include, but are not too limited to, Escherichia coli, Salmonella, Shigella, Pseudomonas, Moraxella, Helicobacter, Stenotrophomonas, Bdellovibrio, Legionella, Chlamydia and Yersinia. Preferably, the attenuated bacterium is a live attenuated bacterium.

Preferably, the genetically engineered microorganism may be a Salmonella spp. Examples of Salmonella species for use in the present invention are Salmonella enterica and Salmonella bongori, preferably Salmonella enterica is used. Salmonella enterica can be further sub-divided into different serotypes or serovars. Examples of said serotypes or serovars for use in the present invention are Salmonella enterica Typhi, Salmonella enterica Paratyphi A, Salmonella enterica Paratyphi B, Salmonella enterica Paratyphi C, Salmonella enterica Typhimurium and Salmonella enterica Enteritidis. In a preferred embodiment, the genetically engineered microorganism is Salmonella enterica serovar Typhi or Salmonella enterica serovar Typhimurium. It is envisaged that any attenuated, non-pathogenic, Salmonella enterica serovar Typhi/Typhimurium strain may be used as herein described, examples of such strains include, but are not limited to, Ty21a, CVD 908-htrA, CVD 909, Ty800, M01ZH09 (also referred to as ZH9), x9633, x9640, x8444, ZH9PA, DTY88, MD58, WTO5, ZH26, SL7838, SL7207, VNP20009 or A1-R. Preferably, the Salmonella enterica serovar Typhi strain is a Salmonella enterica serovar Typhi ZH9 strain.

The present invention discloses a genetically engineered microorganism that has been engineered to comprise the modified autotransporter construct herein described. Accordingly, the present invention discloses a genetically engineered microorganism that may be used for heterologous protein delivery in a subject. Said genetically engineered microorganisms have been mutated in order to provide attenuated strains, for example, bacterial strains, that are effective heterologous protein carriers and delivery systems, whilst maintaining an acceptable safety profile.

As would be understood by a person of skill in the art, genes may be mutated by a number of well-known methods in the art, such as homologous recombination with recombinant plasmids targeted to the gene of interest. In which case an engineered gene with homology to the target gene is incorporated into an appropriate nucleic acid vector (such as a plasmid or a bacteriophage), which is transfected into the target cell. The homologous engineered gene is then recombined with the natural gene to either replace or mutate it to achieve the desired inactivated mutation. Such modification may be in the coding part of the gene or any regulatory portions, such as the promoter region. As would be understood by a person of skill in the art, any appropriate genetic modification technique may be used to mutate the genes of interest, such as the CRISPR/Cas system, e.g. CRISPR/Cas 9.

Thus, numerous methods and techniques for genetically engineering bacterial strains will be well known to the person skilled in the art. These techniques include those required for introducing heterologous genes into the bacteria either via chromosomal integration or via the introduction of a stable autosomal self-replicating genetic element. Exemplary methods for genetically modifying (also referred to as “transforming” or “engineering”) bacterial cells include bacteriophage infection, transduction, conjugation, lipofection or electroporation. A general discussion on these and other methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); which are hereby incorporated by reference.

In a preferred embodiment, the genetically engineered microorganism may be derived from a Salmonella species and comprises an attenuating mutation in a Salmonella Pathogenicity Island 2 (SPI-2) gene and an attenuating mutation in a second gene. Suitable genes and details of such a live attenuated bacterium is as described in WO 2009/158240, which is hereby incorporated by reference in its entirety.

In one embodiment, the SPI-2 gene may be an ssa gene. For example, the invention includes an attenuating mutation in one or more of ssaV, ssaJ, ssaU, ssaK, ssaL, ssaM, ssaO, ssaP, ssaQ, ssaR, ssaS, ssaT, ssaD, ssaE, ssaG, ssal, ssaC and ssaH. Preferably, the attenuating mutation is in the ssaV or ssaJ gene. Even more preferably, the attenuating mutation may be in the ssaV gene.

The genetically engineered microorganism may also comprise an attenuating mutation in a second gene, which may or may not be in the SPI-2 region. The mutation may be outside of the SPI-2 region and involved in the biosynthesis of aromatic compound. For example, in one embodiment, the invention includes an attenuating mutation in an aro gene. In a preferred embodiment, the aro gene may be aroA or aroC. Even more preferably, the aro gene is aroC.

In a preferred embodiment, the genetically engineered microorganism comprising the autotransporter construct herein disclosed is a Salmonella enterica serovar Typhi or Typhimurium strain. In a most preferred embodiment, the genetically engineered microorganism comprising the autotransporter construct herein disclosed is a Salmonella enterica serovar Typhi ZH9 strain. Accordingly, the present invention also discloses a Salmonella enterica serovar Typhi, a Salmonella enterica serovar Typhimurium or a Salmonella enterica serovar Typhi ZH9 strain comprising a modified EstA, MisL, Hbp, AIDA-1, EstP or Pet autotransporter, said modified autotransporters being capable of carrying and secreting heterologous cargo that cross the inner and outer membrane of the Salmonella enterica serovar Typhi/Typhimurium/Typhi ZH9 strain. Whilst it is understood that said strain may carry and secrete any heterologous cargo, in a preferred embodiment, the heterologous cargo is a cytokine, a chemokine, a cytotoxic agent or a cancer antigen.

In another preferred embodiment, the genetically engineered microorganism may be derived from the Salmonella enterica serovar Typhi, wherein said strain comprises a modification in which the lipopolysaccharide O2 O-antigens of Salmonella enterica serovar Paratyphi A is expressed. In yet another preferred embodiment, the genetically engineered microorganism is derived from the Salmonella enterica serovar Typhi, wherein said strain comprises a modification in which the flagella proteins of Salmonella enterica serovar Paratyphi A are expressed. In some instance the genetically engineered microorganism may be derived from the Salmonella enterica serovar Typhi, wherein said strain comprises a modification in which both the lipopolysaccharide O2 O-antigens and the flagella proteins of Salmonella enterica serovar Paratyphi A are expressed, i.e. is the ZH9PA strain.

The live attenuated strain, according to above, may have its native fliC gene replaced with the fliC gene of Salmonella enterica serovar Paratyphi A, such that the conferred serotype is altered from an Hd serotype to a Ha serotype, where ‘serotype’ refers to a distinct variation within the bacterial species. Details of such a modification can be found in WO2020/157203.

The live attenuated strain described above may be further modified to contain a functional fepE gene, such that long 0-antigen chains are generated, preferably wherein the 0-antigen chains are 100 repeated units of the trisaccharide backbone in length. Details of such a modification can be found in WO2020/157203.

It is further envisaged that the live attenuated strain described above may be modified to constitutively express gtrC or to express gtrC in trans. Details of such a modification can be found in WO2020/157203.

It is further envisaged that the live attenuated strain described above may be further modified to contain an additional copy of the tviA gene under the control of a phagosomally induced promoter. Details of such a modification can be found in WO2020/157203.

In a third aspect, the present invention provides for a vaccine composition comprising the modified autotransporter herein disclosed. Accordingly, the present invention also provides for a vaccine composition comprising the genetically engineered microorganism herein disclosed, wherein the genetically engineered microorganism acts as a carrier for said modified autotransporter.

In one embodiment, the vaccine composition may further comprise an adjuvant, pharmaceutically acceptable carrier or excipient.

As used herein, “pharmaceutically acceptable carrier/adjuvant/diluent/excipient” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Examples include, but are not limited to disodium hydrogen phosphate, soya peptone, potassium dihydrogen phosphate, ammonium chloride, sodium chloride, magnesium sulphate, calcium chloride, sucrose, borate buffer, sterile saline solution (0.9% NaCl) and sterile water.

Suitable aqueous and non-aqueous carriers that may be employed in the vaccine compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The vaccine compositions herein disclosed may further contain adjuvants such as preservatives, wetting agents, emulsifying agents and dispersing agents. Prevention of presence of unwanted microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminium monostearate and gelatin. The vaccine composition may also optionally include additional therapeutic agents, known to be efficacious in, for example, infectious disease or neoplastic disease. Accordingly, the vaccine composition herein disclosed may also comprise antiretroviral drugs, antibiotics, antifungals, antiparasitics and chemotherapy drugs.

The vaccine composition may also comprise additional components intended for enhancing an immune response in a subject following administration. Examples of such additional components include but are not limited to; aluminium salts such as aluminium hydroxide, aluminum oxide and aluminium phosphate, oil-based adjuvants such as Freund's Complete Adjuvant and Freund's Incomplete Adjuvant, mycolate-based adjuvants (e.g., trehalose dimycolate), bacterial lipopolysaccharide (LPS), peptidoglycans (e.g., mureins, mucopeptides, or glycoproteins such as N-Opaca, muramyl dipeptide [MDP], or MDP analogs), proteoglycans (e.g., extracted from Klebsiella pneumoniae), streptococcal preparations (e.g., OK432), muramyldipeptides, Immune Stimulating Complexes (the “Iscoms” as disclosed in EP 109 942, EP 180 564 and EP 231 039), saponins, DEAE-dextran, neutral oils (such as miglyol), vegetable oils (such as arachis oil), liposomes, polyols, the Ribi adjuvant system (see, for instance, GB-A-2 189 141), vitamin E, Carbopol, interferons (e.g., IFN-alpha, IFN-gamma, or IFN-beta) or interleukins, particularly those that stimulate cell mediated immunity (e.g., IL-2, IL-3, IL-4, IL-5, IL-6, IL-7, IL-8, IL-9, IL-10, IL-1 1, IL-12, IL-13, IL-14, IL-15, IL-16 and IL-17).

The vaccine composition may be given in isolation or in combination with additional therapies in a concurrent/simultaneous manner or alternatively, completely separately. The terms “concurrent” and “simultaneous” refers to administration of said therapies occurring on the same day. The term “separately” refers to administration of said therapies occurring on different days.

In a fourth aspect, the present invention provides for an immunotherapeutic composition comprising the modified autotransporter herein disclosed. Accordingly, the present invention also provides for an immunotherapeutic composition comprising the genetically engineered microorganism herein disclosed, wherein the genetically engineered microorganism acts as a carrier for said modified autotransporter. In such cases, the cargo of the modified autotransporter may encode for a cancer antigen, or other proteins able to stimulate and/or enhance an immune response in a subject, for example, enhancing the T-cell response. Examples of such other proteins include cytokines, chemokines, or cytotoxic agents. Examples of such cytokines and chemokines include, but are not limited to GM-CSF, IL-2, IL-3, IL-4, IL-5, IL-6, IL-7, IL-8, IL-9, IL-10, IL-11, IL-12, IL-13, IL-14, IL-15, IL-16 and IL-17, Il-21, IFNα, IFNβ, IFNγ, TNFα, CXCL9, CXCL10, CCL5 and MIP1α.

In one embodiment, the immunotherapeutic composition may further comprise a checkpoint inhibitor, an antigen-specific T cell, a therapeutic antibody, a cancer vaccine or other cellular component of the immune system.

The immunotherapeutic composition may comprise a blocking agent directed against an immune checkpoint. The blocking agent may be an antagonist, an inhibitor or a blocking antibody. Accordingly, the blocking agent may be a small molecule or a biologic drug, in particular instances it is a monoclonal antibody. In a preferred embodiment the checkpoint inhibitor is directed against CTLA-4, PD-1, PD-L1, LAG-3, TIM-3, BTLA, TIGIT, VISTA or any combinations thereof. For example, the immunotherapeutic composition may comprise checkpoint inhibitors directed against PD-1 and PD-L1, PD-1 and CTLA-4, PD-L1 and CTLA-4.

Even more preferably, the checkpoint inhibitor is directed against CTLA-4, PD-1 or PD-L1. In some instances, the blocking agent may be ipilimumab (Yervoy®-targeting CTLA-4), nivolumba (Opdivo®-targeting PD-1), pembrolizumab (Keytruda®-also targeting PD-1), atezolizumab (Tecentriq®-targeting PD-L1) or durvalumab (Imfinzi®-targeting PD-L1).

The PD-L1/PD-1 signalling pathway is a primary mechanism of cancer immune evasion for several reasons. First, and most importantly, this pathway is involved in negative regulation of immune responses of activated T effector cells, found in the periphery. Second, PD-L1 is up-regulated in cancer microenvironments, while PD-1 is also up-regulated on activated tumour infiltrating T cells, thus possibly potentiating a vicious cycle of inhibition. Third, this pathway is intricately involved in both innate and adaptive immune regulation through bi-directional signalling. These factors make the PD-1/PD-L1 complex a central point through which cancer can manipulate immune responses and promote its own progression. As a result, the tumour is able to activate inhibitory immune checkpoint molecule pathways, resulting in a suppressed immune system and the continued unimpeded growth of cancerous cells. Following T-cell activation, CTLA-4 is transported to the surface where it competes with CD28 for the same ligands as on the antigen-presenting cells (APCs), resulting in suppression of CD28 and subsequent suppression of T-cell activation and proliferation. Targeting PD-1, PD-L1 and CTLA-4 aims to prevent these events from occurring.

The immunotherapeutic composition may comprise an antigen-specific T cell, wherein the antigen-specific T cell is a result of adoptive T cell therapy. By ‘adoptive T cell therapy’, we intend the transfer of T cells into a subject. The T cells may have originated from the subject (autologous) or from another subject (allogeneic). Examples of such adoptive T cell therapies include, but are not limited to Tumour-Infiltrating Lymphocyte (TIL) therapy, Engineered T Cell Receptor (TCR) therapy and Chimeric Antigen Receptor (CAR) T Cell therapy. It is particularly envisaged that the adoptive T cell therapy may be CAR-T cell therapy. In some instances, the CAR-T cell therapy will be directed against the antigen CD19, which is present in B-cell derived cancers. Accordingly, such therapy may be particularly suited for B-cell derived cancers, such as acute lymphoblastic leukemia (ALL) and diffuse large B-cell lymphoma (DLBCL). In other instances the CAR-T cell therapy will be directed against tumour-associated antigens (TAAs) and are accordingly more suited for the treatment of solid tumours. Examples of such antigens include, but are not limited to, CD133, CD138, CEA, EGFR, EpCAM, GD2, GPC3, HER2, HerinCAR-PD1, MSLN, MG7, MUC1, LMP1, BCMA, PSMA and PSCA. Such techniques will be known to those skilled in the art and the reader is directed to the review entitled “Adoptive cellular therapies: the current landscape” for further information (Rohaan et al. 2019).

The immunotherapeutic composition may comprise a therapeutic antibody directed at the cancer or tumour. In particular embodiments, the therapeutic antibody may be a monoclonal antibody, and even more preferred, a humanised or human monoclonal antibody. Methods of obtaining such monoclonal antibodies are known to those skilled in the art. The therapeutic antibody may block an abnormal protein in a cancer cell or attach to specific proteins on cancer cells. The latter flags the cancer cells to the immune system so that the abnormal cells can subsequently be targeted and destroyed by cellular components of the immune system. In some instances, the monoclonal antibody may also be a checkpoint inhibitor. For example, ipilimumab (Yervoy®), nivolumba (Opdivo®) and pembrolizumab (Keytruda®) are all monoclonal antibodies as well as checkpoint inhibitors. Examples of non-checkpoint inhibitor monoclonal antibodies for the treatment of cancer include, but are not limited to, trastuzumab (Herceptin®), bevacizumab (Avastin®), cetuximab (Erbitux®), panitumumab (Vectibix®), rituximab (Rituxan® and Mabthera®), alemtuzumab (Campath®), ofatumumab (Arzerra®), gemtuzumab ozogamicin (Mylotarg®) and brentuximab vedotin (Adcetris®).

The immunotherapeutic composition may comprise a cancer vaccine. The cancer vaccine may be a preventative vaccine or a treatment vaccine, preferably the vaccine is a treatment vaccine. The use of cancer vaccines boost the immune system's ability to recognise and destroy the antigens presented by the cancerous cells. Such vaccines may also comprise adjuvants to help boost the response even further. Similar to the adoptive T cell therapy, the cancer vaccine may be either autologous or allogeneic.

The immunotherapeutic composition may comprise any other cellular component of the immune system, which may be suitable for use in immunotherapy.

It is understood that the immunotherapeutic composition may include one or more of the immune therapies described above in addition to the modified autotransporter herein described. For example, adoptive T cell therapy and checkpoint inhibitors may be used in combination.

In a fifth aspect, the present invention provides for the vaccine composition or immunotherapeutic composition herein disclosed for use in the prophylactic or therapeutic treatment of an infectious disease or a neoplastic disease.

In one embodiment, the infectious disease may be a viral infection, a bacterial infection, a fungal infection and/or a parasitic infection. In one embodiment, the viral infection may be caused by a coronavirus. Examples of coronaviruses include SARS-CoV-2, MERS-CoV and SARS-CoV. In a preferred embodiment, the viral infection is caused by SARS-CoV-2. In another embodiment, the bacterial infection may be caused by Yersinia pestis, enterotoxigenic E. coli, Clostridium difficile and/or Chlamydia trachomatis. The skilled person will readily understand that any microorganism antigen may be introduced into the modified autotransporter herein disclosed, and thus be appropriate for the treatment of a range of different infectious diseases, for example, an infection caused by a virus, bacteria, fungi or parasite. In one embodiment, the neoplastic disease may be associated with a cancer selected from prostate cancer, liver cancer, renal cancer, lung cancer, colorectal cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer or sarcoma. In a preferred embodiment, the neoplastic disease is associated with a cancer selected from lung cancer, bladder cancer, gastric cancer, ovarian cancer, colorectal cancer, head and neck cancer, melanoma, renal cancer or breast cancer. However, the present invention is envisaged to be suitable for a wide range of cancers. As such, neoplasia, tumours and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. Cancers that may be treated according to the invention include but are not limited to cells or neoplasms of the bladder, blood, bone, bone marrow, brain, breast, colon, esophagus, gastrointestines, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumour, malignant; thecoma, malignant; granulosa cell tumour, malignant; androblastoma, malignant; Sertoli cell carcinoma; Leydig cell tumour, malignant; lipid cell tumour, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumour; Mullerian mixed tumour; nephroblastoma; hepatoblastoma; carcinosarcoma; mesenchymoma, malignant; Brenner tumour, malignant; phyllodes tumour, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumour of bone; Ewing's sarcoma; odontogenic tumour, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant; neurofibrosarcoma; neurilemmoma, malignant; granular cell tumour, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Preferably, the neoplastic disease may be tumours associated with a cancer selected from prostate cancer, liver cancer, renal cancer, lung cancer, breast cancer, colorectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma. The tumour may be metastatic or a malignant tumour.

It is envisaged, that the present invention may therefore be used to achieve a therapeutic benefit in the context of infectious disease and cancer. Specific non-limiting examples of therapeutic benefit include a reduction in viral load of the virus in question, a reduction in the symptoms associated with said virus, a reduction neoplasia, tumour or cancer, or metastasis volume (size or cell mass) or numbers of cells, inhibiting or preventing an increase in neoplasia, tumour or cancer volume (e.g., stabilizing), slowing or inhibiting neoplasia, tumour or cancer progression, worsening or metastasis, or inhibiting neoplasia, tumour or cancer proliferation, growth or metastasis.

The vaccine composition and immunotherapeutic composition herein disclosed will typically be administered to the subject in a composition that comprises an effective amount of the genetically engineered microorganism comprising the modified autotransporter and further comprises a pharmaceutically acceptable carrier/adjuvant/diluent or excipient, the definitions of which are described above.

It is preferred that the vaccine composition and the immunotherapeutic composition comprising the genetically engineered microorganism, and thus the modified autotransporter, herein disclosed may be administered orally, however, it is also contemplated that other methods of administration may be used in some cases. Therefore, in certain instances the genetically engineered microorganism of the present invention may be administered by injection, infusion, continuous infusion, intravenously, intradermally. intraarterially, intraperitoneally, intralesionally, intravitreally, intravaginally, intrarectally, topically, intratumourally, intramuscularly, intraperitoneally, subcutaneously, subconjunctival, intravesicularlly, mucosally, intrapericardially, intraumbilically, intraocularally, intracranially, intraarticularly, intraprostaticaly, intrapleurally, intratracheally, intranasally, topically, locally, inhalation (e.g. aerosol inhalation), via a catheter, via a lavage, or by other method or any combination of the forgoing as would be known to one of ordinary skill in the art (see, for example, Remington's Pharmaceutical Sciences, 18th Ed. Mack Printing Company, 1990).

It is envisaged that administration of the vaccine or immunotherapeutic composition according to the present invention would be carried out following an appropriate dosing regimen. The term “appropriate dosing regimen” is to be construed as a schedule or timescale of one or more administrations of the compositions of the present invention, which may resultantly yield the most effective results in consideration of efficacy and safety of the subject to which the composition is being administered. For example, the compositions herein disclosed may comprise a single dose or multiple doses, for example, two or doses. In some instances, a subsequent dose may be given following the first dose, approximately three weeks later to the first dose. It is envisaged that the dose of genetically engineered microorganism comprising the modified autotransporter herein disclosed may be in the range 10⁵ to 10¹² CFU, preferably in the range of 10⁹ to 10¹⁰ CFU, wherein CFU is the colony-forming unit. The skilled person will be familiar with the use of such units. CFU is a unit used to estimate the number of viable microorganisms in a sample. For example, suitable doses may be between 10⁵ and 10⁶ CFU, 10⁵ and 10⁷ CFU, 10⁵ and 10⁸ CFU, 10⁵ and 10⁹ CFU, 10⁵ and 10¹⁰ CFU, 10⁵ and 10¹¹ CFU, 10⁶ and 10⁷ CFU, 10⁶ and 10⁸ CFU, 10⁶ and 10⁹ CFU, 10⁶, and 10¹⁰ CFU, 10⁶ and 10¹¹ CFU, 10⁶ and 10¹² CFU, 10⁷ and 10⁸ CFU, 10⁷ and 10⁹ CFU, 10⁷ and 10¹⁰ CFU, 10⁷ and 10¹¹ CFU, 10⁷ and 10¹² CFU, 10⁸ and 10⁹ CFU, 10⁸ and 10¹⁰ CFU, 10⁸ and 10¹¹ CFU, 10⁸ and 10¹² CFU, 10⁹ and 10¹⁰ CFU, 10⁹ and 10¹¹ CFU, 10⁹ and 10¹² CFU, 10¹⁰ and 10¹¹ CFU, 10¹⁰ and 10¹² CFU, or 10¹¹ and 10¹² CFU.

There exists further the possibility to further administer boost dosages after a more extended period of time. This may be selected as an appropriate measure if a subject's immunoglobulin G (IgG) antibody levels or T-cell response fall below determined protective levels. Thus in some embodiments, an appropriate dosage regimen may be given as a “booster”. It is envisaged that such a booster may be given either on an annual basis or every 5-10 years as is deemed necessary.

In a sixth aspect, the present invention provides for a method for modifying a Gram-negative bacterial autotransporter, comprising: i) removing the passenger domain from the passenger region, ii) introducing a synthetic polynucleotide sequence that encodes a first polypeptide tag in the passenger region flanked by restriction enzyme recognition sequences, iii) introducing a synthetic polynucleotide sequence that encodes a second polypeptide tag within the passenger region upstream of the synthetic polynucleotide sequence that encodes the first polypeptide tag, and is positioned outside of the Type IS restriction enzyme recognition sequence boundaries iv) introducing a synthetic polynucleotide sequence that encodes a linker, said sequence being downstream of the synthetic polynucleotide sequence that encodes the first polypeptide tag and is positioned outside of the restriction enzyme recognition sequence boundaries, and v) introducing a synthetic polynucleotide sequence that encodes for a cleavage site within the passenger region downstream of the synthetic polynucleotide sequence that encodes for a linker.

The skilled person will recognise that as the second polypeptide tag and the linker are positioned outside of the restriction enzyme recognition sequence boundaries, they are not removed when performing techniques such Golden Gate cloning and therefore remain on the autotransporter.

In preferred embodiments, the first polypeptide tag may be a FLAG tag or other tag that allows for in-frame translation of the autotransporter, the second polypeptide tag may be a His tag, the linker may be a serine-glycine linker and the cleavage site may be a caspase-3 cleavage site or OmpT cleavage site.

The present invention also provides a modified autotransporter for use in the delivery of a cargo, wherein the modified autotransporter has not yet had the heterologous polynucleotide sequence introduced. Accordingly, the present invention also provides for a method of delivering a cargo molecule, or polypeptide, by inserting the modified autotransporter, with or without the polynucleotide sequence encoding for said cargo molecule, into a microorganism.

The inventors of the present invention have surprisingly discovered a way in which to modify known autotransporters so that heterologous cargo can easily, efficiently and reliably be delivered to a subject/patient. The invention is further described with reference to the following non-limiting examples:

### EXAMPLES

Six different autotransporters were identified from the literature that had previously been used to deliver heterologous cargo. For each of the autotransporters, the sequence encoding the passenger region was replaced with a “placeholder” sequence. Said placeholder sequence containing 1) a first polypeptide tag to maintain structural and sequence integrity, 2) a restriction site to allow for high throughput cloning of different cargo, and optionally, 3) a flexible linker to isolate the cargo from the translocation unit, and 4) an ompT cleavage site next to a Casp-3 cleavage site.

Once the autotransporters were synthesised, they were subsequently cloned into a plasmid with pBR322 on (˜15-20 copies/cell) and downstream of ssaG promoter (ssaGp), previously shown to be macrophage-inducible (induced inside the Salmonella containing vacuole) and active on the artificial media Pseudomonas-CN-agar (PCN).

Cargoes were cloned using Golden Gate reaction using standard reaction conditions.

### Example 1

### Validation of Cargo Carrying Autotransporters

Validation experiments were performed to confirm the secretion of the heterologous cargo of the modified autotransporters herein described. The beta-lactamase gene (bla) was selected as the test cargo to demonstrate successful secretion. The beta-lactamase gene encodes an enzyme that opens the lactam ring of the antibiotic ampicillin, rendering it ineffective and thus preventing bacterial killing. It is known that beta-lactamase is only effective when translocated to the periplasm, as it is unable to come into contact with the antibiotic if it remains in the cytosol, and that it is unable to cross the inner membrane when the signal peptide is removed.

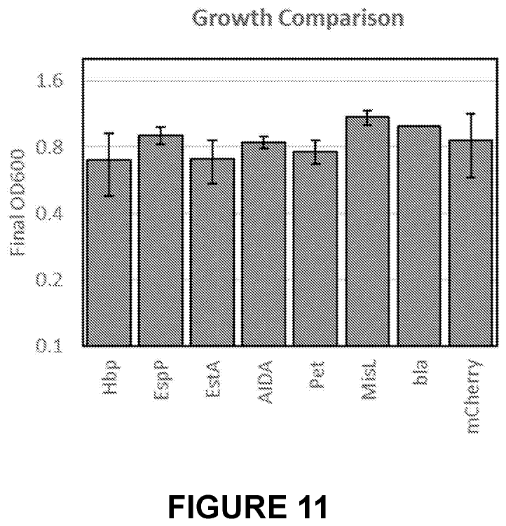

The modified autotransporters encoding the beta-lactamase protein were Hbp, EspP, EstA, AIDA-1, MisL and Pet. It was found that Gram-negative bacterial strains, for example, Salmonella enterica serovar Typhi ZH9, expressing these modified autotransporters resulted in no significant growth defects, compared to the empty vector and bla alone controls (see FIG. 11), thus suggesting that incorporation of the modified autotransporters into such strains does not effect the viability of said strains.

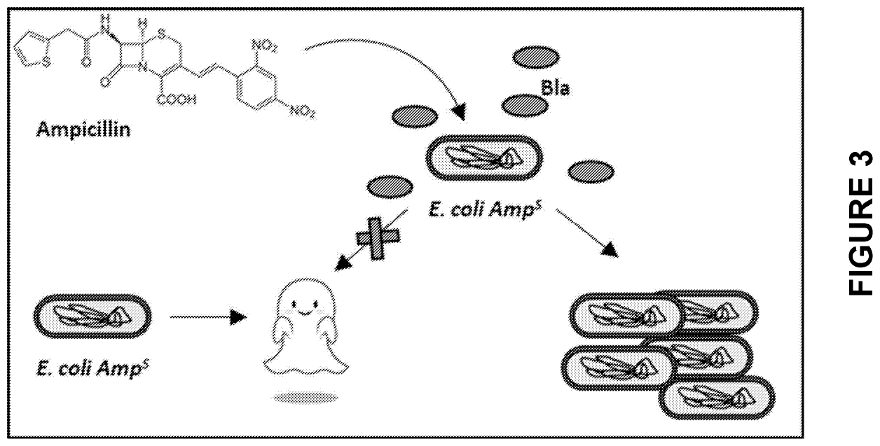

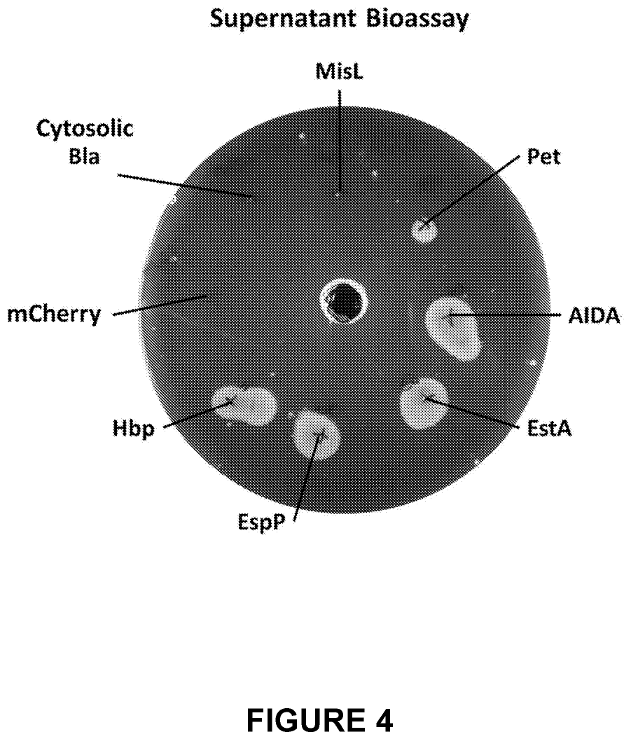

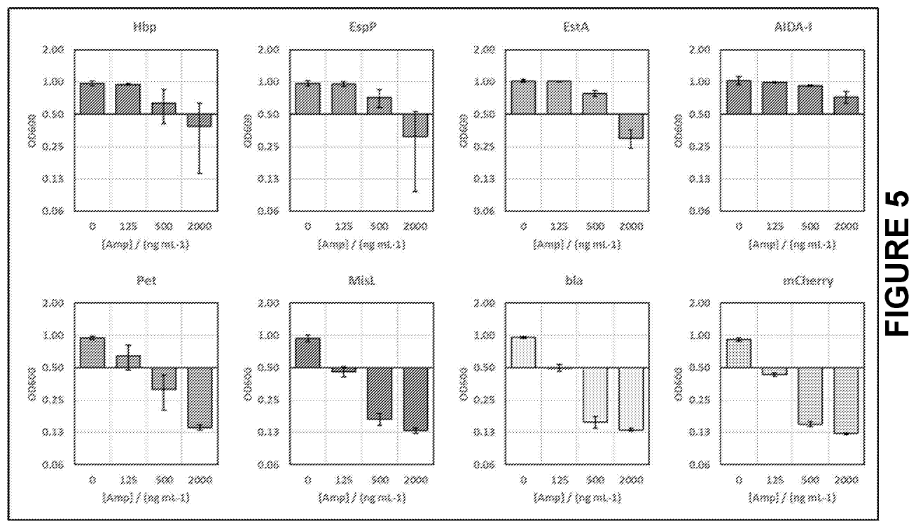

An ampicillin survival challenge assay using concentrated supernatant of each cultured strain was also performed (see FIG. 3). Such an assay involves challenging an indicator strain (for example, E. coli) with the antibiotic ampicillin. In normal conditions, the cells would not survive, whereas, if beta-lactamase enzyme is present in the supernatant, the antibiotic is degraded and thus the cells are allowed to grow. Importantly, the more beta-lactamase there is, the higher the concentration of ampicillin the cells will be able to sustain, and therefore survive. Accordingly, such an assay allows the ranking of the modified autotransporters for their ability to export heterologous cargo, for example, beta-lactamase. It was found that the modified autotransporters Hbp, EstP, EstA and AIDA-1 are able to successfully secrete heterologous cargo, i.e. beta-lactamase into the culture supernatant, whereas Pet and MisL provided modest secretion (possibly due to a lack of cleavage site in MisL). See FIGS. 4 and 5.

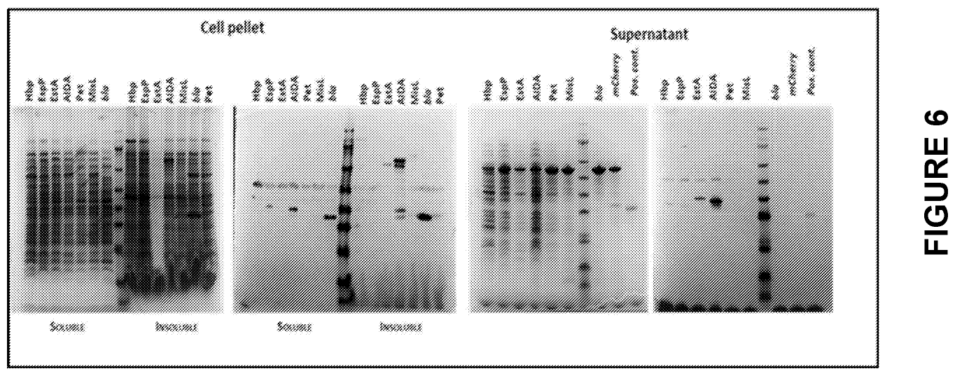

Secretion of beta-lactamase was also identified via Western Blot in some cases, for example, wherein the modified autotransporter was a modified EstA or AIDA autotransporter (see FIG. 6). Consistent with previously collected results (not shown here), the 6× Histidine tag (i.e. the second polypeptide tag) was not detectable by Western Blot in 3 out of 6 autotransporters. Without being bound by theory, this is thought to be due to interactions with structure or the signal peptide.

### Example 2

Assessing secretion mechanism in modified strains

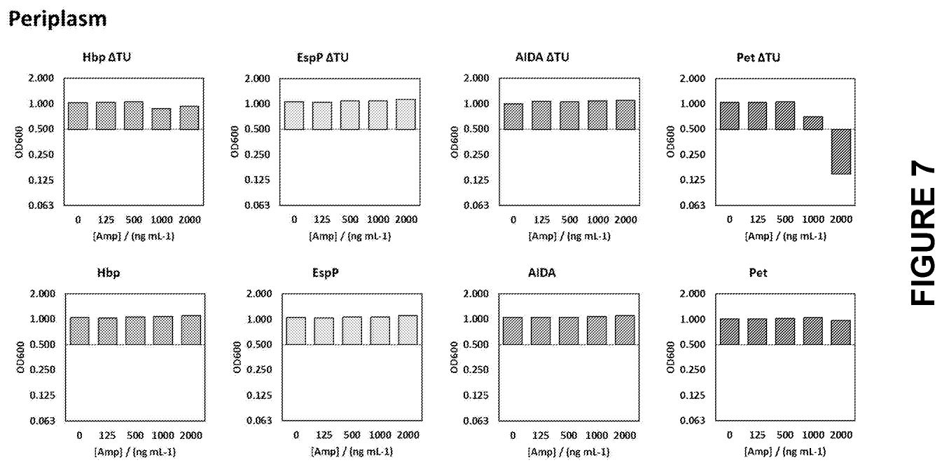

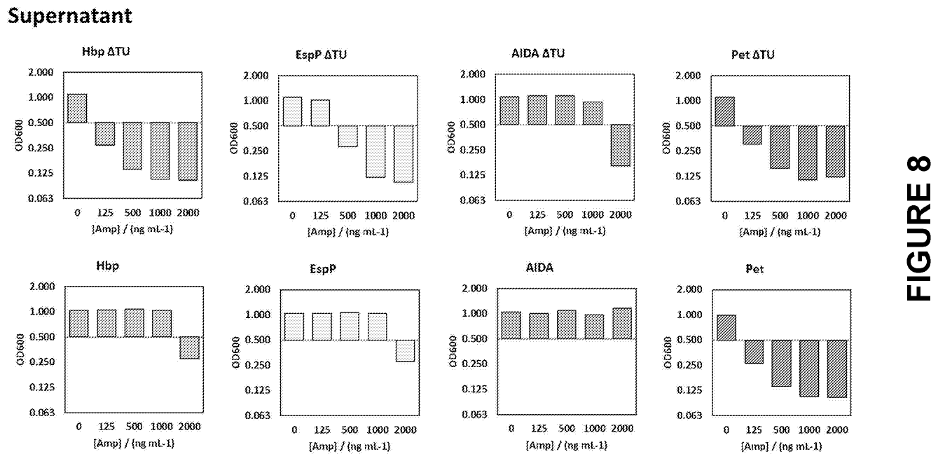

Further experiments were conducted to determine whether translocation of the cargo into the periplasm is condition enough to achieve secretion, i.e. via passive secretion, or whether the translocation unit of the autotransporter(s) is required to export the cargo, i.e. active secretion. Beta-lactamase levels in the culture supernatant, and in the periplasm, following truncation of the translocation unit of four of the modified autotransporters (Hbp, EspP, AIDA, and Pet) were assessed (see FIGS. 7 and 8). The generated data showed that even though some beta-lactamase secretion is achieved upon deleting the translocation unit (consistent with the observation that the outer membrane displays a degree of porosity in Salmonella strains), it appears that the translocation unit is a requisite to achieve successful secretion of beta-lactamase (see FIGS. 7 and 8).

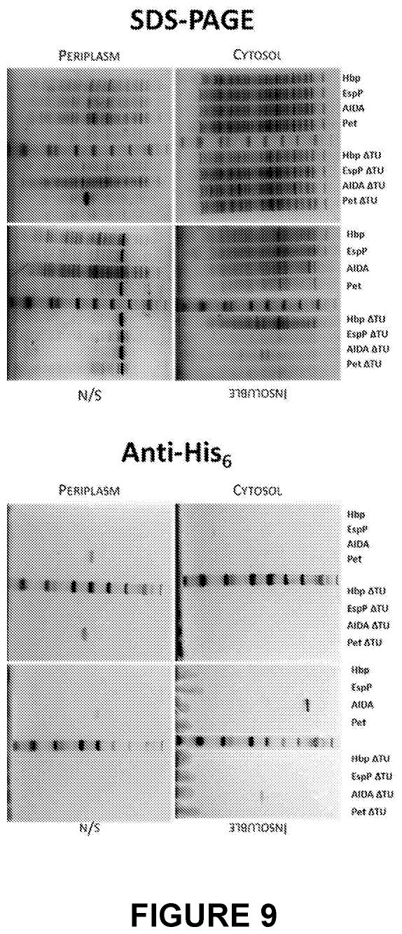

In agreement with these results, when analysed via Western Blotting targeting the 6× Histidine tag, no cargo was detected in the spent PCN media of variants of the autotransporters where the translocation unit was deleted, where it was detected in full length AIDA-I, and in the periplasm of the truncated AIDA- and full length AIDA-b (see FIG. 9).

### Example 3

Assessing secretion of various cargo proteins Finally, further experiments were conducted to assess if the autotransporters were able to export any desired cargo. As a proof-of-concept study, the V antigen from Yersinia pestis was selected to exemplify the secretion of an antigenic cargo, and the secretion of cytokine IL-18 was chosen as an example of how the present invention can be applied in the immuno-oncology field. The DNA sequences encoding for these proteins were designed to contain the required restriction sites (see FIG. 2) and introduced as passengers into all the autotransporters. The plasmids were introduced into ZH9 and expressed in PCN medium for 5 h at 37° C.

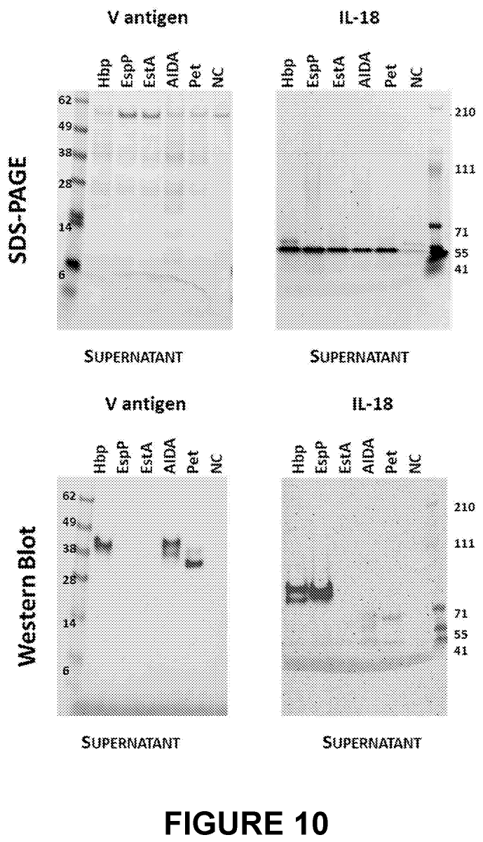

The spent media was then concentrated and assessed for the presence of cargo via Western Blot, using cargo-specific antibodies (Thermofisher MA1-23088 for V antigen, Abcam ab191152 for IL-18) (see FIG. 10). The results show that in both examples the cargo was successfully exported in more than one AT.

Accordingly, the results herein show successful export of the desired cargo into the supernatant and demonstrate the suitability of the autotransporter construct herein disclosed in a variety of applications, for example, in the vaccine and immunotherapy fields.

1. An autotransporter construct modified to permit insertion of a heterologous polynucleotide sequence that encodes a target polypeptide for translocation across the inner and outer membrane of a Gram-negative bacterium, the autotransporter comprising i) a polynucleotide sequence that encodes a N-terminal signal sequence; ii) a passenger region into which said heterologous polynucleotide sequence encoding the target polypeptide is to be inserted and iii) a polynucleotide sequence encoding a translocation domain; wherein the passenger region comprises a synthetic polynucleotide sequence flanked by restriction enzyme recognition sequences, wherein said synthetic polynucleotide sequence comprises a polynucleotide sequence that encodes a first polypeptide tag.

2. The autotransporter construct of claim 1, wherein the passenger region further comprises a synthetic polynucleotide sequence that encodes a linker, preferably wherein the linker is a serine-glycine linker.

3. The autotransporter construct of claim 1, wherein the passenger region further comprises a synthetic polynucleotide sequence that encodes a second polypeptide tag, preferably wherein the second polypeptide tag is a His tag.

4. The autotransporter construct of claim 1, wherein the passenger region further comprises a synthetic polynucleotide sequence that encodes a cleavage site, preferably wherein the cleavage site is a caspase-3 cleavage site and/or an OmpT cleavage site.

5. The autotransporter construct of claim 1, wherein the synthetic polynucleotide sequence that encodes the second polypeptide tag is upstream of the synthetic polynucleotide sequence that encodes the first polypeptide tag.

6. The autotransporter construct of claim 1, wherein the first polypeptide tag is any synthetic polynucleotide sequence that allows for the in-frame translation of the autotransporter, preferably the first polypeptide tag is a FLAG tag.

7. The autotransporter construct of claim 1, wherein the synthetic polynucleotide sequence that encodes the second polypeptide tag is located upstream of the synthetic polynucleotide sequence that encodes the first polypeptide tag, the synthetic polynucleotide sequence that encodes the linker is located downstream of the synthetic polynucleotide sequence that encodes the first polypeptide tag and the synthetic polynucleotide sequence that encodes the cleavage site is located downstream of the synthetic polynucleotide sequence that encodes the linker.

8. The autotransporter construct of claim 1, wherein the autotransporter to be modified is EstA, MisL, Hbp, AIDA-1, EstP or Pet.

9. The autotransporter construct of claim 1, wherein the autotransporter construct further comprises a heterologous polynucleotide encoding a target peptide or protein.

10. The autotransporter construct of claim 1, wherein the heterologous polynucleotide encodes for a therapeutic protein.

11. The autotransporter construct of claim 9, wherein the heterologous polynucleotide encodes for an anti-cancer therapeutic or immunogenic molecule.

12. The autotransporter construct of claim 11, wherein the anti-cancer therapeutic or immunogenic molecule is a cytokine, a chemokine, an antibody or a fragment thereof, a cytotoxic agent, a cancer antigen, or any combination thereof.

13. The autotransporter construct of claim 9, wherein the heterologous polynucleotide encodes for IFNα, IFNβ, IL-18.

14. A genetically engineered microorganism comprising the autotransporter construct of claim 1.

15. The genetically engineered microorganism of claim 14, wherein the genetically engineered microorganism is an attenuated Gram-negative bacterium.

16. The genetically engineered microorganism of claim 14, wherein the genetically engineered microorganism is a Salmonella spp, preferably Salmonella enterica, even more preferably Salmonella enterica serovar Typhi or Salmonella enterica serovar Typhimurium.

17. The genetically engineered microorganism of claim 14, wherein the genetically engineered microorganism is derived from Salmonella spp. and comprises an attenuating mutation in a Salmonella Pathogenicity Island 2 (SPI-2) gene and an attenuating mutation in a second gene, preferably wherein the second gene is an aro gene.

18. The genetically engineered microorganism of claim 14, wherein the genetically engineered microorganism is Salmonella enterica serovar Typhi ZH9.

19. The genetically engineered microorganism of claim 14, wherein the genetically engineered microorganism is derived from Salmonella enterica serovar Typhi, wherein said strain comprises a modification in which the lipopolysaccharide 02 O-antigens and/or the flagella proteins of Salmonella enterica serovar Paratyphi A are expressed.

20. A vaccine composition comprising the autotransporter construct of claim 1.

21. The vaccine composition of claim 20, wherein the vaccine composition further comprises an adjuvant, pharmaceutically acceptable carrier or excipient.

22. A method of prophylactic or therapeutic treatment of an infectious disease or a neoplastic disease in a subject, the method comprising administering to the subject the vaccine composition of claim 20.

23. The method of claim 22, wherein the infectious disease is a viral infection, a bacterial infection, a fungal infection and/or a parasitic infection.

24. The method of claim 22, wherein the neoplastic disease is associated with a cancer selected from prostate cancer, liver cancer, renal cancer, lung cancer, colorectal cancer, bladder cancer, breast cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer or sarcoma.

25. An immunotherapeutic composition comprising the modified autotransporter of claim 1.

26. The immunotherapeutic composition of claim 25, wherein the immunotherapeutic composition is given in combination with a checkpoint inhibitor, an antigen-specific T cell, a therapeutic antibody, a cancer vaccine or other cellular component of the immune system.

27. A method of prophylactic or therapeutic treatment of an infectious disease in a subject, the method comprising administering to the subject the immunotherapeutic composition of claim 25.

28. The method of claim 27, wherein the infectious disease is a viral infection, a bacterial infection, a fungal infection and/or a parasitic infection.

29. A method of prophylactic or therapeutic treatment of a neoplastic disease in a subject, the method comprising administering to the subject the immunotherapeutic composition of claim 25.

30. The method of claim 29, wherein the neoplastic disease is associated with a cancer selected from prostate cancer, liver cancer, renal cancer, lung cancer, breast cancer, colorectal cancer, bladder cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer or sarcoma.

31. A method for modifying a Gram-negative bacterial autotransporter, comprising: i) removing the passenger domain from the passenger region, ii) introducing a synthetic polynucleotide sequence that encodes a first polypeptide tag in the passenger region flanked by restriction enzyme recognition sequences, iii) introducing a synthetic polynucleotide sequence that encodes a second polypeptide tag within the passenger region upstream of the synthetic polynucleotide sequence that encodes the first polypeptide tag and is positioned outside of the restriction enzyme recognition sequence boundaries, iv) introducing a synthetic polynucleotide sequence that encodes a linker, said sequence being downstream of the synthetic polynucleotide sequence that encodes the first polypeptide tag and is positioned outside of the restriction enzyme recognition sequence boundaries, and v) introducing a synthetic polynucleotide sequence that encodes for a cleavage site within the passenger region downstream of the synthetic polynucleotide sequence that encodes for a linker.

32. The method of modifying a Gram-negative bacterial autotransporter of claim 31, wherein the first polypeptide tag is a FLAG tag or any other tag that allows in-frame translation of the autotransporter.

33. The method of modifying a Gram-negative bacterial autotransporter of claim 31, wherein the second polypeptide tag is a His tag.

34. The method of modifying a Gram-negative bacterial autotransporter of claim 31, wherein the linker is a serine-glycine linker.

35. The method of modifying a Gram-negative bacterial autotransporter of claim 31, wherein the cleavage site is a caspase-3 cleavage site and/or an OmpT cleavage site.


SEQUENCE LISTING (28 sequences)

SEQ ID NO: 1 - protein, 8 aa, Artificial Sequence
  FLAG tag
DYKDDDDK

SEQ ID NO: 2 - protein, 7 aa, Artificial Sequence
  Restriction enzymes sites
GGTCTCN

SEQ ID NO: 3 - protein, 7 aa, Artificial Sequence
  Restriction enzymes sites
CGTCTCN

SEQ ID NO: 4 - protein, 8 aa, Artificial Sequence
  Restriction enzymes sites
GAAGACNN

SEQ ID NO: 5 - protein, 5 aa, Artificial Sequence
  Linker
GGGGS

SEQ ID NO: 6 - protein, 8 aa, Artificial Sequence
  Linker
GGGGGGGG

SEQ ID NO: 7 - protein, 6 aa, Artificial Sequence
  Linker
GGGGGG

SEQ ID NO: 8 - protein, 12 aa, Artificial Sequence
  Linker
GSAGSAAGSGEF

SEQ ID NO: 9 - protein, 36 aa, Artificial Sequence
  Linker
GGSAGGSGSGSSGGSSGASGTGTAGGTGSGSGTGSG

SEQ ID NO: 10 - protein, 5 aa, Artificial Sequence
  Linker
EAAAK

SEQ ID NO: 11 - protein, 61 aa, Artificial Sequence
  Linker
AEAAAKAEAAAKAEAAAKAEAAAKALEAEAAAKALEAEAAAKALEAEAAAKALEAEAAAKA

SEQ ID NO: 12 - protein, 12 aa, Artificial Sequence
  Linker
AEAAAKEAAAKA

SEQ ID NO: 13 - protein, 5 aa, Artificial Sequence
  Linker
PAPAP

SEQ ID NO: 14 - protein, 10 aa, Artificial Sequence
  Linker
APAPAPAPAP

SEQ ID NO: 15 - protein, 17 aa, Artificial Sequence
  Linker
VSQTSKLTRAETVFPDV

SEQ ID NO: 16 - protein, 6 aa, Artificial Sequence
  Linker
PLGLWA

SEQ ID NO: 17 - protein, 6 aa, Artificial Sequence
  Linker
RVLAEA

SEQ ID NO: 18 - protein, 10 aa, Artificial Sequence
  Linker
EDVVCCSMSY

SEQ ID NO: 19 - protein, 8 aa, Artificial Sequence
  Linker
GGIEGRGS

SEQ ID NO: 20 - protein, 10 aa, Artificial Sequence
  Linker
TRHRQPRGWE

SEQ ID NO: 21 - protein, 10 aa, Artificial Sequence
  Linker
AGNRVRRSVG

SEQ ID NO: 22 - protein, 9 aa, Artificial Sequence
  Linker
RRRRRRRRR

SEQ ID NO: 23 - protein, 4 aa, Artificial Sequence
  Linker
GFLG

SEQ ID NO: 24 - protein, 36 aa, Artificial Sequence
  Linker
GGSGGGSEGGGSEGGGSEGGGSEGGGSEGGGSGGGS

SEQ ID NO: 25 - protein, 6 aa, Artificial Sequence
  Linker
GSSSSS

SEQ ID NO: 26 - protein, 6 aa, Artificial Sequence
  Linker
SSSSSS

SEQ ID NO: 27 - protein, 6 aa, Artificial Sequence
  Linker
GSSGSS

SEQ ID NO: 28 - protein, 12 aa, Artificial Sequence
  Linker
GSAGSAAGSGEF